(19) United States
(12) Patent Application Publication
(10) Pub. No.: US 2026/0151348 A1
(43) Pub. Date: Jun. 4, 2026

(54) Engineered Umbilical Cord Mesenchymal Stem Cell Exosomes Loaded with siCCR5 for Treating Alzheimer's Disease

(71) Applicant: Hebei Zhongkun Bioengineering Co., Ltd., Baoding (CN)

(72) Inventors: Li Zhong, La Verne, CA (US); Wenjing Zhang, Baoding, Hebei (CN); Miou Zhou, Chino Hills, CA (US)

(21) Appl. No.: 19/408,208

(22) Filed: Dec. 3, 2025

(30) Foreign Application Priority Data
Nov. 7, 2024 (CN) 202411582284X

(51) Int. Cl. A61K 9/50; A61K 31/713; A61P 25/28; C12N 5/0775

(52) U.S. Cl. CPC A61K 9/5068; A61K 31/713; A61P 25/28; C12N 5/0668

(57) ABSTRACT

The invention relates to engineered umbilical cord mesenchymal stem cell exosomes (hUCMSC-EVs) loaded with siCCR5, their preparation method, and their use in treating Alzheimer's disease. A lipid membrane is first prepared and dissolved, followed by incorporation of siCCR5 and hUCMSC-EVs. Using a cationic liposome extrusion technique, siCCR5 is efficiently delivered into the exosomes to obtain siCCR5-loaded engineered EVs. The resulting exosomes promote tissue regeneration, repair brain tissue, and modulate the brain microenvironment without causing toxicity. By carrying siCCR5, which targets a specific gene, the engineered EVs exhibit stronger targeted therapeutic effects and enhanced anti-inflammatory activity compared to conventional hUCMSC-EVs, thereby improving Alzheimer's disease progression.

### TECHNICAL FIELD

The present disclosure relates to the field of biopharmaceutical technology, and more particularly to engineered umbilical cord mesenchymal stem cell exosomes loaded with siCCR5, methods of preparing such engineered exosomes, and their use in the treatment of Alzheimer's disease.

### BACKGROUND

Alzheimer's disease (AD) is a neurodegenerative disease associated with neuronal damage and is a major cause of dementia. Aging leads to loss of protein homeostasis, and the incidence of AD increases with age. In 2019, AD was listed as the sixth leading cause of death in the United States. In addition, COVID-19 (coronavirus disease 2019) has significantly impacted AD-related mortality. It is estimated that by 2060 there will be approximately 13.8 million AD patients in the United States, imposing a tremendous healthcare burden on families and society and adversely affect social development.

Pathologically, AD is characterized by amyloid-β (Aβ) plaques and neurofibrillary tangles (NFTs) formed by hyperphosphorylated tau protein. Soluble amyloid-β oligomers (AβO) can cause neuronal dysfunction and activate neuroglial cells in the brain, thereby inducing chronic neuroinflammation.

The U.S. Food and Drug Administration (FDA) has approved memantine, donepezil, galantamine, and rivastigmine for the treatment of AD. These drugs are cholinesterase inhibitors that can alleviate cognitive impairment by inhibiting acetylcholinesterase-mediated degradation of acetylcholine. However, due to the complexity of AD pathogenesis, there are currently no effective therapies or drugs that can fundamentally cure AD; existing treatments merely relieve symptoms. Therefore, there is an urgent need for new therapeutic strategies for AD.

In recent years, mesenchymal stem cells (MSCs) have been regarded as a novel therapeutic option for AD due to their low immunogenicity, anti-inflammatory effects, and multipotent differentiation capacity, and have entered clinical trials. MSCs may act in neurodegenerative diseases through three main mechanisms: secretion of growth factors for immune modulation, suppression of neuroinflammation, and paracrine release of exosomes. Studies indicate that MSCs exert inhibitory effects on AD primarily via paracrine exosomes rather than the cells themselves. Exosomes contain proteins, lipids, DNA, mRNA, miRNA and other substances, play important roles in cell-to-cell communication, do not cause systemic toxicity, and can promote tissue regeneration to repair brain tissue, modulate the brain microenvironment, and suppress inflammation. Thus, exosomes have broad application prospects in the clinical treatment of AD.

Chemokine C-C motif receptor 5 (CCR5) is a cell membrane protein of the G protein-coupled receptor family and is an important receptor for leukocyte activation and mobilization, including monocyte lineage cells. In the central nervous system, CCR5 is highly expressed in microglia. Activation of CCR5 triggers different signaling cascades that alter calcium flux and chemotaxis, affect proliferation and apoptosis, and promote migration of activated cells, leading to pro-inflammatory cytokine release and stimulation of downstream immune cells. Recent work published in Cell has shown that CCR5 plays a key role in the regulation of learning and memory. Delayed expression of CCR5 in the CA1 region of mice reduces neuronal excitability, closes the time window for memory linking, and causes memory impairment in aged mice. However, to date CCR5 has not been combined with exosomes as a therapeutic strategy for AD.

Therefore, there is a need to modify exosomes so that they can serve as natural carriers to load siCCR5, thereby constructing engineered exosomes capable of gene modulation. Such engineered exosomes can integrate cell therapy and gene therapy and are of great importance for the treatment of AD.

### SUMMARY

The present disclosure aims to provide engineered umbilical cord mesenchymal stem cell exosomes loaded with siCCR5, methods for preparing such engineered exosomes, and their use in the treatment of Alzheimer's disease. The engineered exosomes can be used as therapeutics for AD, achieve combined cell and gene therapy, significantly improve memory, and enhance spatial memory performance in subjects in need thereof.

To achieve the above objectives, the present disclosure provides the following technical solutions.

In one aspect, the present disclosure provides a method of preparing engineered exosomes, comprising the steps of:
(1) preparation of a lipid film: dissolving DLin-MC3-DMA in an organic solvent (chloroform:methanol=9:1, v/v), further dissolving cholesterol and DOPC to prepare a lipid solution; removing the organic solvent by rotary evaporation and drying under an inert gas atmosphere to obtain a lipid film; and
(2) preparation of engineered exosomes: hydrating the lipid film with a phosphate-buffered saline (PBS) solution, sonicating, adding siCCR5 and umbilical cord mesenchymal stem cell-derived exosomes (hUCMSC-EVs) to obtain a mixed solution; allowing the mixed solution to hydrate, and under incubation conditions, performing filtration and extrusion followed by dialysis to obtain engineered exosomes.

In some embodiments, in step (1), the organic solvent is a mixed solvent of chloroform:methanol at a volume ratio of 9:1, and the molar ratio of DLin-MC3-DMA:cholesterol:DOPC is 25-34.5:35-65:1.5-40.

In some embodiments, in step (2), during hydration, the mass-to-volume ratio of lipid film to PBS is 100:3, and the mass ratio of siCCR5:hUCMSC-EVs:lipid film is 2:1:50.

In some embodiments, in step (2), the incubation temperature is 40-50° C.

In some embodiments, in step (2), the siCCR5 is designed to knock down CCR5 and comprises a sense strand having the sequence of SEQ ID NO.1 and an antisense strand having the sequence of SEQ ID NO.2.

In some embodiments, in step (2), the extrusion step comprises:

first extruding the mixed solution through a filter having a pore size of 1.0 μm, then extruding through a filter having a pore size of 0.1 μm, and finally extruding through a filter having a pore size of 0.05 μm.

In some embodiments, the three extrusion steps use polycarbonate membrane filters, and each extrusion step is performed 2-5 times.

In some embodiments, in step (2), the dialysis step comprises:
dialyzing the sample overnight at 4° C. using a dialysis cassette with a molecular weight cut-off of 100 kDa, and adjusting the pH to 7.2-7.4.

In another aspect, the present disclosure provides engineered exosomes prepared by the above method.

In a further aspect, the present disclosure provides the use of the engineered exosomes in the preparation of a medicament for treating Alzheimer's disease.

The engineered exosomes of the present disclosure offer several advantages over the prior art:

In the present disclosure, lipids are used to modify exosomes, and a cationic liposome extrusion method is employed to deliver siCCR5 into exosomes, thereby obtaining umbilical cord mesenchymal stem cell exosomes engineered to carry siCCR5. The engineered exosomes prepared according to the present disclosure do not cause systemic toxicity and can promote tissue regeneration to repair brain tissue, modulate the brain microenvironment, and suppress inflammation. Furthermore, by loading siCCR5 targeting a specific gene, the engineered exosomes exhibit stronger targeting therapeutic efficacy and more efficient anti-inflammatory activity than conventional hUCMSC-Exos, thereby improving the progression of Alzheimer's disease and achieving combined cell and gene therapy to significantly enhance memory and spatial learning.

### BRIEF DESCRIPTION OF THE EXPERIMENTAL FIGURES

To more clearly describe the technical solutions of the embodiments of the present disclosure or of the prior art, illustrative figures included in this specification are briefly described below. These figures form part of the written description and provide experimental data, characterization results, and schematic workflows for understanding the embodiments. It will be understood by those skilled in the art that the figures represent only some embodiments of the present disclosure, and that other figures or variations may be derived without inventive effort.

FIG. 1 is a flow cytometry analysis of cell-surface marker proteins in Example 1.

FIG. 2 shows the morphology of MSC-3P1 cells on day 2 of culture in Example 1.

FIG. 3 shows the flow cytometry analysis of surface markers of umbilical cord mesenchymal stem cells in Example 1.

FIG. 4 shows osteogenic and adipogenic differentiation staining results of umbilical cord mesenchymal stem cells in Example 1.

FIG. 5 shows the ultracentrifugation workflow for isolating exosomes in Example 1.

FIG. 6 is an electron micrograph of hUCMSCs-Exos in Example 1.

FIG. 7 shows the particle size analysis of hUCMSCs-Exos in Example 1.

FIG. 8 shows expression of exosomal marker proteins in hUCMSCs-Exos in Example 1.

FIG. 9 shows miRNA sequencing analysis of hUCMSCs-Exos in Example 1.

FIG. 10 shows the technical route for preparing engineered exosomes in Example 1.

FIG. 11 is an electron micrograph of engineered exosomes in Example 1.

FIG. 12 shows particle size analysis of engineered exosomes in Example 1.

FIGS. 13A-13E shows the effects of hUCMSCs-Exos on reducing CCR5 expression in microglial cells in Test Example 1, wherein FIG. 13A shows differential gene enrichment analysis; FIG. 13B shows the gene expression heatmap; FIG. 13C shows the GO enrichment analysis pathways; and FIGS. 13D-13E show changes in CCR5 expression at the protein and gene levels in BV2 cells after treatment with hUCMSCs-Exos.

FIG. 14 shows the identification of transgenic mice in Test Example 1.

FIG. 15 shows the results of the novel object recognition test and novel location recognition test in APP/PS1 mice in Test Example 1.

FIG. 16 shows the Morris water maze test results of APP/PS1 mice in Test Example 1, wherein (a) shows the escape latency; (b) shows the swimming trajectories; (c) shows the number of platform crossings; (d) shows the time spent in the target quadrant; (e) shows the total swimming distance; and (f) shows the swimming speed of each group.

### DETAILED DESCRIPTION

The following provides a detailed description of various exemplary embodiments of the present disclosure. This detailed description should not be regarded as limiting the present disclosure, but rather as providing a more detailed explanation of certain aspects, features, and embodiments of the disclosure.

The terminology used herein is intended only to describe particular embodiments and is not intended to be limiting. Furthermore, with respect to numerical ranges disclosed herein, it should be understood that each intermediate value between the upper and lower limits of the range is specifically disclosed. Each smaller range between any stated value or intermediate value and any other stated value or intermediate value within the disclosed range is also included within the present disclosure. The upper and lower limits of these smaller ranges may be independently included within or excluded from the range.

Unless otherwise indicated, all technical and scientific terms used herein have the same meaning as commonly understood by those of ordinary skill in the art to which the present disclosure pertains. Although preferred methods and materials are described herein, any methods or materials similar or equivalent to those described herein may also be used in the practice or testing of the present disclosure. All publications cited herein are incorporated by reference for the purpose of disclosing and describing the methods and/or materials associated with the cited publications. In the event of any conflict between an incorporated publication and the present specification, the present specification shall control.

Various modifications and variations can be made to the specific embodiments described herein without departing from the scope or spirit of the present disclosure. Such modifications and variations will be apparent to those skilled in the art in view of the teachings herein. The present specification and the examples provided herein are intended to be illustrative only.

As used herein, the terms “comprise,” “include,” “have,” “contain,” and the like are intended to be open-ended terms, meaning “including but not limited to.”

The present disclosure provides a method for preparing engineered exosomes, comprising the following steps:
(1) Preparation of lipid film: dissolving lipids in an organic solvent, further dissolving cholesterol and DOPC to prepare a lipid solution; removing the organic solvent by rotary evaporation and drying under an inert gas atmosphere to obtain a lipid film.
(2) Preparation of engineered exosomes: hydrating the lipid film with phosphate-buffered saline (PBS), sonicating, and then adding siCCR5 and umbilical cord mesenchymal stem cell-derived exosomes to obtain a mixed solution; hydrating the mixed solution and, under incubation conditions, performing filtration, extrusion, and dialysis to obtain engineered exosomes.

In the present disclosure, siCCR5 is a short nucleic acid sequence designed to knock down CCR5 and includes:

Sense (SEQ ID NO. 1):
GGUCAGUUCCGACCUAUAGTT;
Antisense (SEQ ID NO. 2):
CUAUAGGUCGGAACUGACCTT.

### Example 1

Example 1 provides a method for preparing engineered exosomes, comprising the following steps:

### (1) Isolation and Culture of Umbilical Cord Mesenchymal Stem Cells

### A. Isolation of Primary Umbilical Cord Mesenchymal Stem Cells:

Fresh, healthy umbilical cord tissue was cut into 2-3 cm segments, and the Wharton's jelly portion was dissected out. The Wharton's jelly was then minced and transferred into a culture flask. An appropriate amount of culture medium was added, and the flask was placed in a CO₂ incubator. The flask was not shaken during incubation to avoid detachment of the tissue explants. When the migrated cells covered approximately 90% of the bottom surface of the culture flask, the primary cells were passaged.

### B. Passage Culture of Umbilical Cord Mesenchymal Stem Cells:

When the cells reached approximately 90% confluence, the spent culture medium in the flask was discarded, and an appropriate amount of PBS was added to wash the cells. After removing the PBS, an appropriate amount of gentle stem cell dissociation trypsin was added evenly. When the cells detached from the bottom of the flask, an equal volume of serum-free complete medium was added to terminate digestion. The cell suspension was pipetted up and down to disperse the cells and transferred into a centrifuge tube, followed by centrifugation at 1000 rpm for 5 minutes at room temperature. The supernatant was discarded, and the cells were resuspended in an appropriate amount of complete medium and counted. The cells were then seeded into a clean culture flask containing complete medium, gently shaken to ensure uniform distribution, and placed in a 37° C. CO₂ incubator to complete the passage.

### C. Identification of Umbilical Cord Mesenchymal Stem Cells

### (1) Morphological Identification

The morphology of the cells was observed under a microscope, and the results are shown in FIG. 2.

FIG. 2 shows that the cells exhibited fibroblast-like morphology.

### (2) Identification of Surface Markers

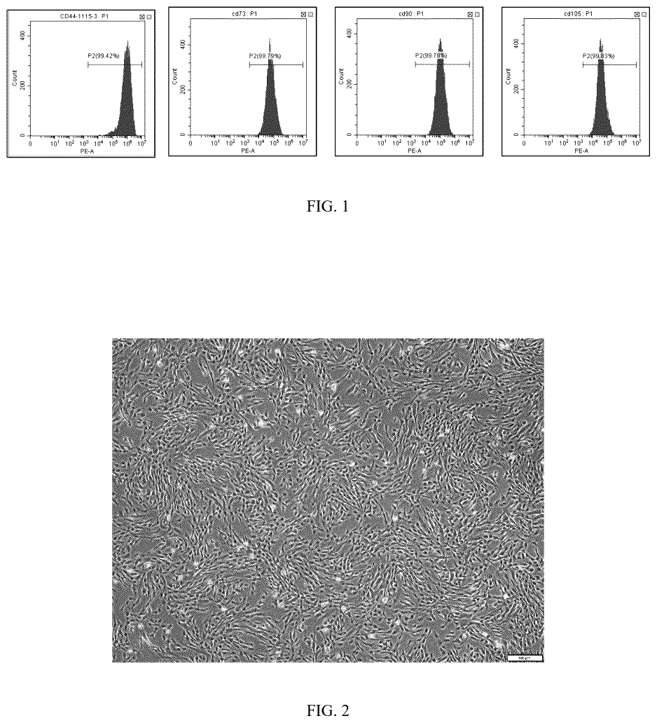

Flow cytometry was used to measure the expression levels of the cell-surface markers CD44, CD73, CD90, CD105, CD34, and CD45, with an IgG isotype control used as the negative control. The results are shown in FIG. 1.

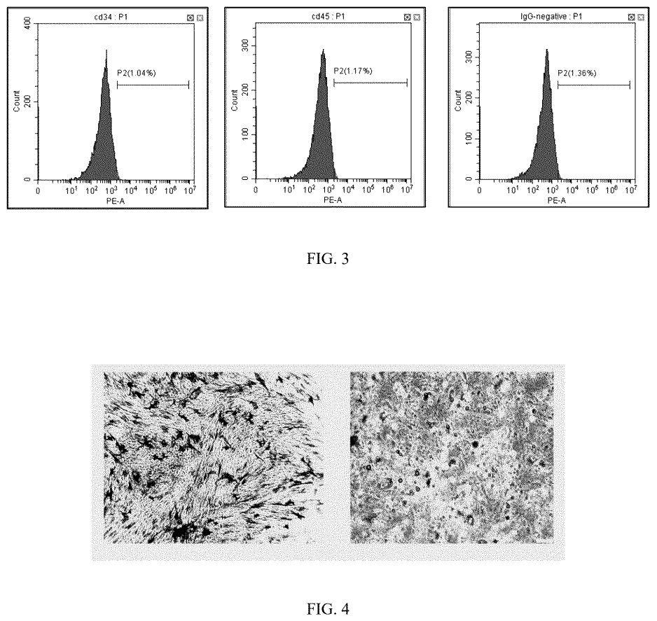

As shown in FIG. 3, the positive MSC markers CD44, CD73, CD90, and CD105 were expressed at levels exceeding 95%, while the negative markers CD34 and CD45 were expressed at levels below 1.0%. These results meet the identification criteria, confirming that the cells were mesenchymal stem cells.

### (3) Identification of Osteogenic and Adipogenic Differentiation of Stem Cells

MSCs possess multipotent differentiation potential, meaning that under specific induction conditions, they can differentiate into osteoblasts and adipocytes. Their in vitro osteogenic and adipogenic differentiation capacity can be evaluated through specific staining.

### Osteogenic Induction and Identification:

Seeding of MSCs: Log-phase mesenchymal stem cells were seeded into six-well plates at a density of 2×10⁴ cells/cm² and cultured at 37° C. in a 5% CO₂ incubator until reaching 60-70% confluence. The supernatant was discarded and induction medium was added. The osteogenic induction medium consisted of DMEM complete medium (DMEM+10% FBS+1% P/S+200 μM sodium L-ascorbate+100 μM β-glycerophosphate+0.1 μM dexamethasone). The adipogenic induction medium consisted of DMEM+10% FBS+1% P/S+1 μM dexamethasone +0.5 mM 3-isobutyl-1-methylxanthine+200 μM indomethacin+10 μg/mL insulin. Differentiation induction: The osteogenic/adipogenic induction medium was replaced every 2-3 days, and cells were cultured at 37° C. in a 5% CO₂ incubator for approximately 14-21 days while observing morphological changes. Based on the cell condition, induction was terminated and staining identification was performed. Cell fixation: The induction medium was removed, and cells were washed once with 1×PBS. After removal of PBS, 2 mL of 4% neutral formaldehyde solution was added to cover the bottom surface of the culture vessel, fixed at room temperature for 30 minutes, followed by two washes with PBS.

Osteogenic Alizarin Red staining: Before use, the staining solution was equilibrated to room temperature. One milliliter of 1% Alizarin Red solution (pH 4.2) was added to each well and incubated at 37° C. for 30 minutes. The staining solution was removed, and cells were washed twice with PBS, followed by the addition of PBS to prevent drying. Adipogenic Oil Red O staining: Oil Red O working solution was prepared by mixing Oil Red O stock solution with ddH₂O at a ratio of 3:2, followed by filtration through a membrane. The working solution was freshly prepared prior to use. One milliliter of the working solution was added to each washed well and incubated at 37° C. for 30 minutes. The staining solution was removed, and cells were washed twice with PBS, followed by the addition of PBS to prevent drying.

Induction assessment: The osteogenic and adipogenic staining results were examined under a microscope, and images were collected for evaluation. Successful osteogenic induction was indicated by red or orange-red calcium nodules formed by binding of Alizarin Red. Osteogenic nodules appeared purple, whereas lipid droplets formed after adipogenic induction were stained red, as shown in FIG. 4.

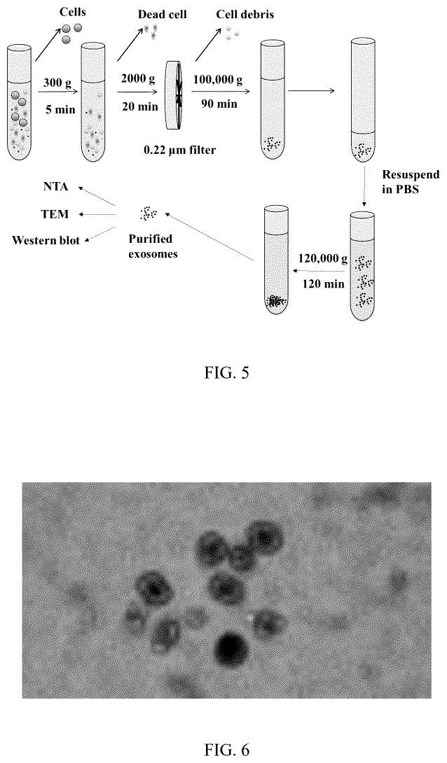

(2) Isolation of Umbilical Cord Mesenchymal Stem Cell-Derived Exosomes (hUCMSCs-EVs)
A. The MSCs obtained from step (1) were expanded, and the cells were processed according to the centrifugation scheme shown in FIG. 5. Passage-cultured cells were centrifuged at 300×g for 5 minutes to remove cells, followed by centrifugation at 2000×g for 20 minutes. The supernatant was then filtered through a 0.22 μm membrane, resuspended in PBS, and centrifuged at 120,000×g for 120 minutes to obtain the exosomes (hUCMSCs-EVs).

### B. Identification of Exosomes

### (1) Transmission Electron Microscopy

The isolated exosomes were examined using transmission electron microscopy (TEM), and the results are shown in FIG. 6.

FIG. 6 shows that the hUCMSCs-EVs exhibited a typical cup-shaped morphology with a bilayer membrane structure.

### (2) Exosome Particle Size

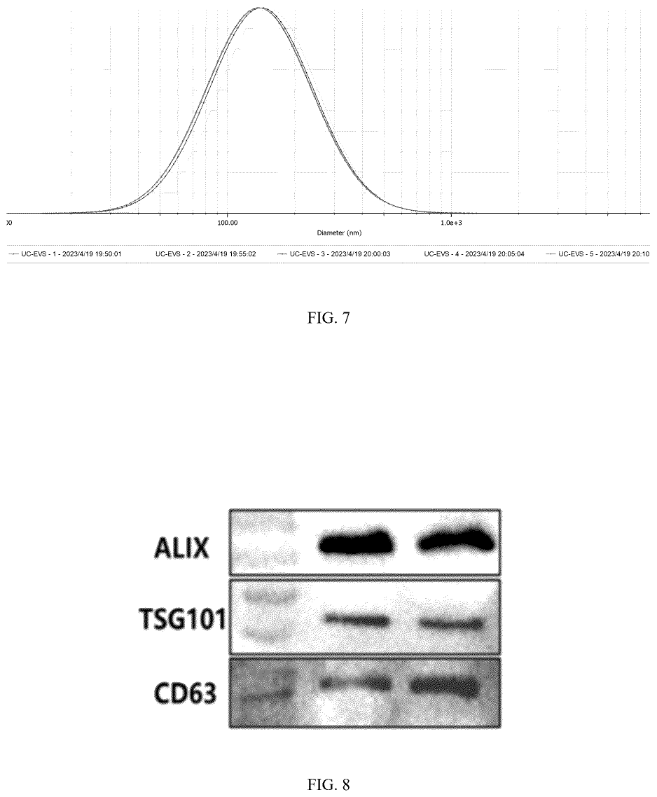

The particle size of the isolated exosomes was measured, and the results are shown in FIG. 7.

FIG. 7 shows that five repeated nanoparticle analyses indicated an exosome diameter distribution around 100 nm, which is consistent with the known size range of exosomes.

### (3) Western Blot Analysis of Exosomal Marker Proteins

TSG101, CD63, and ALIX proteins were selected as exosomal marker proteins. TSG101 is associated with the ESCRT complex. CD63 is a member of the tetraspanin family and is directly involved in ESCRT-dependent and ESCRT-independent sorting of extracellular vesicle cargo. ALIX is directly involved in the membrane scission process during the formation of vesicles that bud off from the plasma membrane to form independent membrane structures. Therefore, the expression of these proteins can be used to identify exosomes. The results are shown in FIG. 8.

FIG. 8 shows that the extracted exosomes expressed all three marker proteins.

(4) High-Throughput Sequencing Analysis of miRNAs in hUCMSCs-EVs

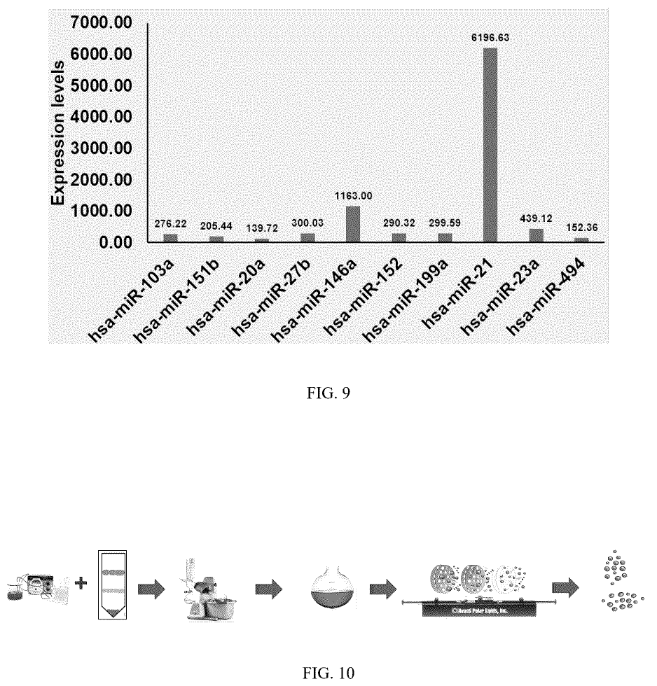

miRNAs are key functional components within exosomes. In the present disclosure, the types and abundances of miRNAs in the exosomes were analyzed by high-throughput sequencing. The results are shown in FIG. 9.

FIG. 9 shows that highly abundant miRNAs include miR-21, miR-146, and others, which are associated with various signaling pathways, regulate downstream target gene expression, and affect cellular metabolism. At present, extensive research has focused on miRNAs as therapeutic targets in a variety of metabolism-related diseases.

### (3) Construction of Engineered Exosomes

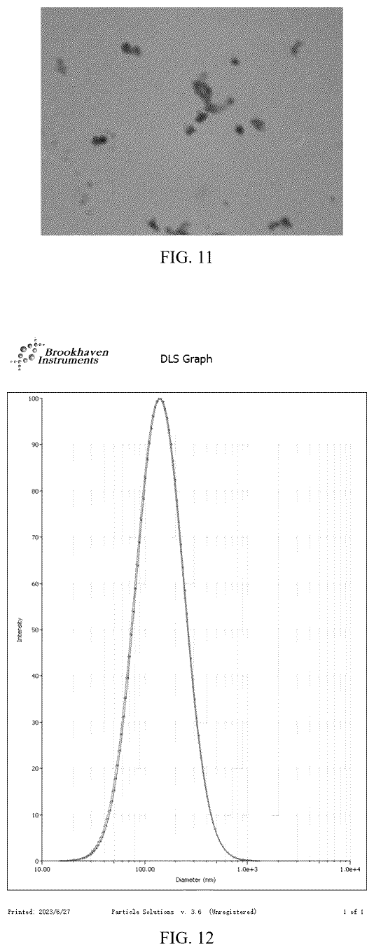

Engineered exosomes were prepared according to the procedures shown in FIGS. 10 and 11, as described below.
A. Preparation of lipid solution: lipids were dissolved in a chloroform/methanol mixture (9/1, v/v) and formulated into a lipid solution at a molar ratio of 25:35:40 (DLin-MC3-DMA:DOPC:cholesterol) with a final amount of 4 nmol and a total volume of 1 mL.

DLin-MC3-DMA was purchased from MCE Bio; DOPC was purchased from Macklin Reagent Co.; and cholesterol was purchased from Sima Biotechnology Co.
B. Formation of lipid film by rotary evaporation: the prepared lipid solution was transferred into a 5-mL round-bottom flask. At 45° C., the organic solvent was evaporated under vacuum using a rotary evaporator, and the resulting lipid film was dried under nitrogen for approximately 20 minutes.
C. Hydration and extrusion: the lipid film was hydrated with phosphate-buffered saline (PBS) (reagents and ratios as shown in FIG. 2), followed by sonication for 5 minutes. A mixture of siCCR5 (purchased from Suzhou Gemma Gene Co.) and umbilical cord mesenchymal stem cell-derived exosomes at a mass ratio of 2:1 was added, and the suspension was fully hydrated at a mass ratio of siCCR5:exosomes:totallipid film of 2:1:50. The suspension was maintained at 45° C. and then sequentially extruded five times through polycarbonate membrane filters with pore sizes of 1.0 μm, 0.1 μm, and 0.05 μm using a hand extruder. The sample was subsequently dialyzed overnight at 4° C. using a 100-kDa molecular weight cut-off dialysis cassette (Slide-A-Lyzer G210K, regenerated cellulose membrane 22.5-30 μm), adjusting the pH to 7.4 and removing unencapsulated siRNA to obtain engineered exosomes.

### (4) Characterization of Engineered Exosomes

A. Transmission electron microscopy (TEM) analysis of the engineered exosomes was performed, and the results are shown in FIG. 12.

FIG. 12 shows that the hybrid particles exhibit a quasi-spherical, typical “cup-shaped” morphology, with sizes mainly in the range of 30-150 nm. These results are consistent with the morphological characteristics of hybrid particles, and the TEM images confirm successful hybridization of lipid nanoparticles, exosomes, and siCCR5.

### B. Particle Size of the Engineered Exosomes

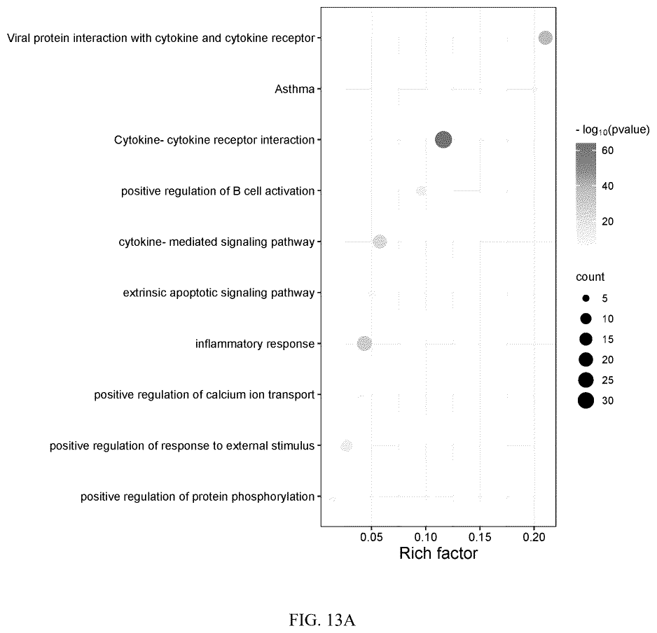

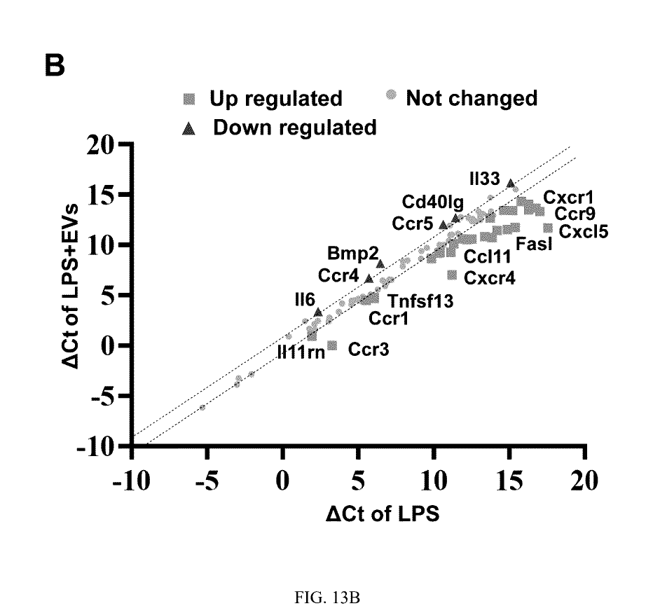

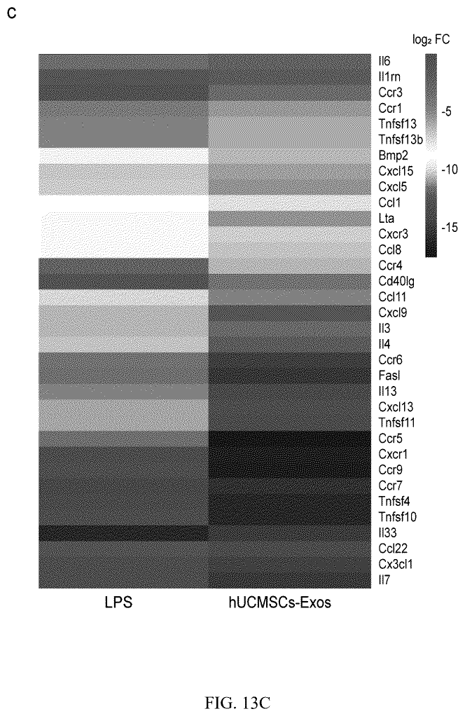

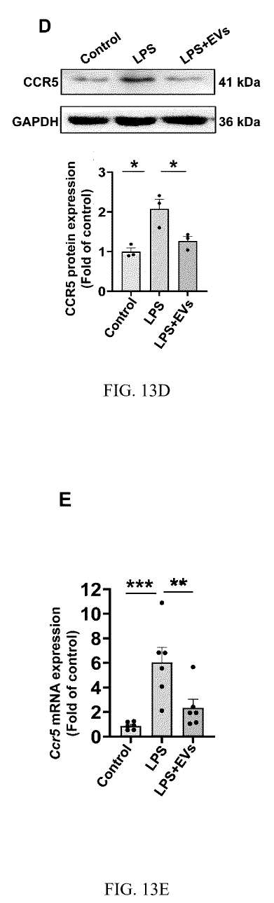

Because the hybrid particles are formed from exosomes, siRNA, and lipid nanoparticles—each having different particle sizes-nanoparticle size analysis was performed to assess sample purity. The results are shown in FIG. 13.

FIG. 13 shows that the engineered exosomes exhibit a relatively uniform particle size distribution, with a diameter primarily in the 90-100 nm range, consistent with expected particle size characteristics.

### Example 2

Example 2 provides another method for preparing engineered exosomes. The difference from Example 1 is that in Example 2, the mass ratio of exosomes to total lipid content is 1:25.

### Test Example 1

Test Example 1 evaluated the therapeutic effects of the engineered exosomes prepared in Example 1 on Alzheimer's disease. The specific methods are as follows:

### (1) Establishment and Analysis of the Cell Model

Because Alzheimer's disease is closely associated with microglial cells, the deposition of amyloid plaques in the brain triggers inflammatory responses in microglia, leading to the release of multiple inflammatory cytokines and migration toward the plaques for phagocytic clearance, thereby inducing an immune response. Therefore, in the present disclosure, lipopolysaccharide (LPS) was used to establish an inflammatory microglial cell model.

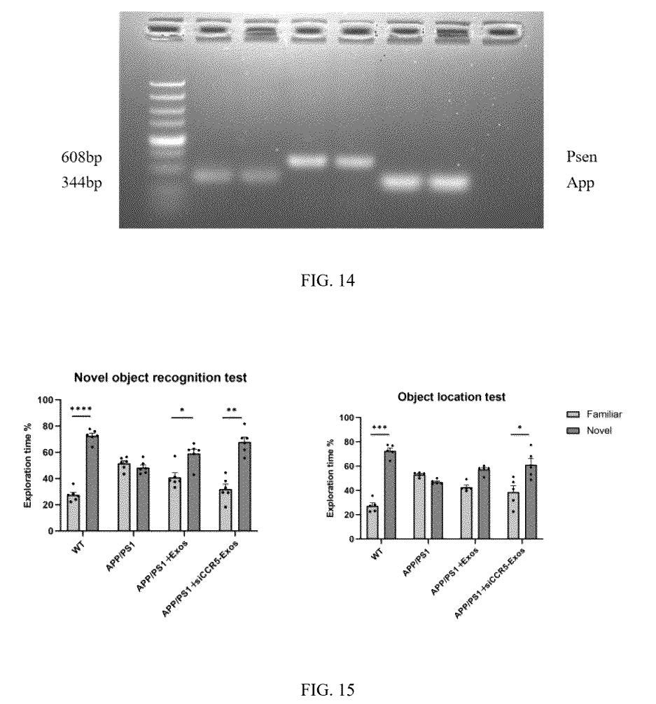

BV2 microglial cells were seeded into six-well plates at a density of 1×10⁵ cells per well and divided into three groups: a normal control group, an LPS-induced inflammation group, and an EV-treated group. After the cells adhered for 12 hours, both the LPS group and the EV-treated group were treated with LPS at 1 μg/mL for 24 hours. After 24 hours, the medium for the EV-treated group was replaced with basal medium and supplemented with hUCMSC-EVs at a concentration of 20 μg/mL for an additional 24 hours. The results are shown in FIG. 14.

The present disclosure analyzed the expression of inflammatory cytokines using qPCR. FIG. 14 shows that treatment with LPS at 1 μg/mL for 24 hours significantly increased inflammatory cytokine expression. After exosome treatment, inflammatory cytokine expression was markedly reduced, indicating that the exosomes inhibited the inflammatory response of microglia. In addition, treatment with hUCMSC-EVs enhanced the migratory capacity of microglia, facilitating movement toward amyloid deposition sites for clearance.

Sequencing analysis performed in the present disclosure demonstrated that after exosome treatment, the expression of CCR5 in microglial cells was significantly reduced at both the gene and protein levels (FIG. 14, panels b-d), confirming that umbilical cord mesenchymal stem cell-derived exosomes are associated with CCR5, and that CCR5 is closely related to microglial inflammatory responses (FIG. 14, panel a). Therefore, the present disclosure aims to use stem cell-derived exosomes as carriers to load siCCR5 for application in the treatment of Alzheimer's disease.

### (2) Establishment and Evaluation of a Mouse Model

### (1) Animal Grouping

APP/PS1 transgenic mice were used as the experimental animals. These mice carry five familial AD-related mutations, including three APP mutations and two PSI mutations. The mice exhibit behavioral and pathological characteristics of human AD, including amyloid deposition in the hippocampal and cortical regions and excessive activation of glial cells. Because APP/PS1 transgenic mice are heterozygous, male APP/PS1 mice were crossed with female C57BL/6 wild-type mice. According to Mendelian inheritance, approximately one-fourth of the offspring are expected to be APP/PS1 transgenic mice; therefore, genotyping was required. In this study, PCR was performed using primers for the APP and PSEN genes on DNA extracted from the tail of one-month-old pups. The presence of positive bands indicated successful identification of transgenic model mice, as shown in FIG. 15.

As shown in Table 1, the experimental animals were divided into four groups: wild-type (WT), AD+PBS, AD+hUCMSC-Exos, and AD+siCCR5-engineered hUCMSC-Exos, with six mice in each group. Exosomes were administered intranasally at a dose of 50 mg/kg once per week for four consecutive weeks. Differences before and after exosome administration were observed and recorded.

TABLE 1
lists the grouping and treatment conditions for the in vivo experiments.

| | Number | | | Injection | |
|---|---|---|---|---|---|
| Group | of Mice | Reagent | Dose | Frequency | Duration |
| WT | 5 | Physiological saline | 15 μL | Twice per week | 4 weeks |
| AD | 5 | Physiological saline | 15 μL | Twice per week | 4 weeks |
| AD-Exos | 5 | hUCMSC-Exos | 30 μg | Twice per week | 4 weeks |
| | | | (15 μL) | | |
| AD- | 5 | hUCMSC- | 30 μg | Twice per week | 4 weeks |
| siCCR5Exos | | siCCR5Exos | (15 μL) | | |

### (2) Novel Object Recognition Test

(1) Prior to testing, mice were habituated to the experimenter by gentle handling each day to minimize stress during the procedure.
(2) During the habituation phase, mice were allowed to freely explore the testing apparatus (without objects) for 10 minutes.
(3) During the familiarization phase, two identical objects (A and B) were placed in the apparatus (ensuring that the objects had no odor and were fixed in place). The objects were positioned approximately 10 cm from each side wall to allow sufficient exploration space.
(4) Each mouse was placed into the apparatus at an equal distance from the objects, with its back facing the objects. A camera and software were used to record the exploration time for each object within 5 minutes (exploration was defined as touching the object with the mouth or nose, or approaching within approximately 2-3 cm).
(5) After 24 hours, the test phase was conducted by replacing one of the identical objects with a novel object (AC). Each mouse was again placed into the apparatus at an equal distance from the objects with its back facing the objects, and exploration time for each object within 5 minutes was recorded.

### (3) Novel Location Recognition Test

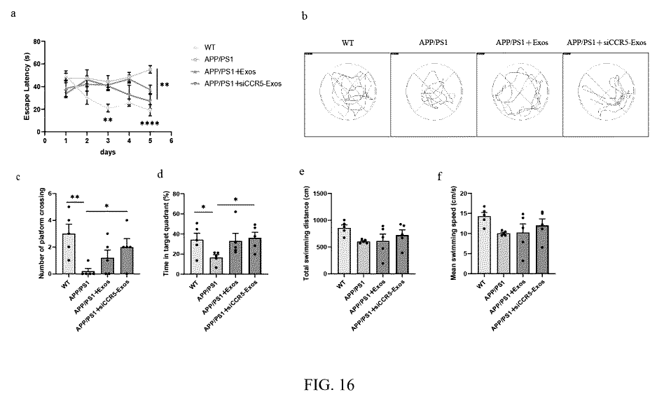

In the training phase of the novel object recognition experiment, the identical objects were placed on the same side of the apparatus. For the novel location recognition test, the training phase remained unchanged, but during the test phase the two objects were placed in diagonal positions. Each mouse was placed into the apparatus at an equal distance from the objects with its back facing the objects, and a camera and software were used to record the exploration time for each object location within 5 minutes. The results are shown in FIG. 16.

FIG. 16 shows that in both the novel object recognition and novel location recognition tests, AD model mice spent relatively little time exploring the novel object (or novel location), with exploration times similar to those for the familiar object (or familiar location). In contrast, the Exos group and the siCCR5-Exos group showed significantly increased exploration of the novel object (or novel location), with the siCCR5-Exos group exhibiting an even more pronounced effect. These results indicate that stem-cell-derived exosomes significantly improved memory performance in mice, and that engineered siCCR5-loaded exosomes produced a superior memory-enhancing effect.

### (4) Morris Water Maze Test

A. Mice were first trained before the test:
(1) In the water maze training, the platform was positioned at the center of the pool, 1 cm below the water surface, allowing the animals to detect its presence. The water temperature was maintained at 26° C.
(2) Each mouse underwent three consecutive training trials. The mouse was placed on the platform for 20 seconds.
(3) The water maze contains four quadrants. The animal was placed into one of the quadrants. The mouse was gently placed into the water tail-first while being supported by hand. The head was not submerged first to avoid causing stress.
(4) The mouse was allowed to swim to locate the platform, with a maximum allowed time of 60 seconds. Initially, mice may swim along the pool wall searching for an exit. Eventually, the mice learn to find and climb onto the platform.
(5) Once a mouse reached the platform, timing was stopped and the latency recorded. If the mouse failed to find the platform within 60 seconds, a latency of 60 seconds was recorded. If the mouse could not reach the platform, it was not picked up; instead, a glass or plastic rod was used to guide it to the platform, where it remained for 15 seconds. The same procedure was repeated for two additional quadrants, with the starting direction changed for each trial.
(6) After completing all three trials, the mouse was dried with a towel. All mice underwent the same series of three training trials, using consistent starting directions, and the latency times were recorded.
B. After the animals completed five days of training, the Morris water maze test was performed.
(1) Before starting the experiment, the pool was filled with tap water and heated, and the platform was positioned 1 cm below the water surface. Skimmed milk, 125 mL of non-toxic white temporary dye, or food coloring was added to make the water opaque.
(2) Lighting and water temperature were kept consistent with the training conditions.
(3) Each mouse was monitored until it reached the platform, and the latency time was recorded. If the mouse did not reach the platform within 60 seconds, the handler guided it to the platform as in training. The mouse remained on the platform for 10 seconds, was then dried, and returned to its home cage.
(4) Each mouse underwent four trials, being placed into the pool from different quadrants. During each trial, the handler returned to the same designated location. The testing order was: all animals' first trial, all animals' second trial, all animals' third trial, and so on. The inter-trial interval was at least 2 minutes.
(5) For the probe test, the platform was removed. Each mouse was allowed to swim freely for 60 seconds to explore the previous platform location. Water maze software was used to record the swimming trajectories during training and testing for subsequent analysis.
(6) After all probe trials were completed, the mice were dried and the pool was drained.

The results are shown in FIG. 17.

In the Morris water maze test, FIG. 17a shows that on the third day of training, the escape latency of the WT group significantly decreased, and the mice quickly located the platform. On the fourth day, the Exos group showed shortened escape latency; on the fifth day, the siCCR5-Exos group demonstrated an even more pronounced improvement. In the swimming trajectories shown in FIG. 17b, APP/PS1 mice exhibited fewer platform crossings and spent less time in the target quadrant compared to WT mice. Both indices increased in the Exos group, with the siCCR5-Exos group showing a more significant effect. FIGS. 17e and 17f show no significant differences in swimming speed or total swimming distance among the groups, indicating that differences in performance were not due to variations in swimming ability. These results demonstrate that exosomes effectively enhanced spatial memory in mice, and that engineered siCCR5-Exos exhibited superior efficacy compared with unmodified exosomes.

The above description presents preferred embodiments of the present disclosure. It should be understood that those skilled in the art may make various modifications and refinements without departing from the principles of the present disclosure, and such modifications and refinements are intended to fall within the scope of the present disclosure.

1. A method of preparing an engineered umbilical-cord-derived mesenchymal stem cell exosome (hUCMSC-EV) loaded with siCCR5, the method comprising:
(a) preparing a lipid film by dissolving DLin-MC3-DMA, cholesterol, and DOPC in an organic solvent to form a lipid solution, removing the organic solvent, and drying the lipid solution under an inert gas; and
(b) hydrating the lipid film in phosphate-buffered saline (PBS), sonicating the hydrated lipid film, adding siCCR5 and hUCMSC-EVs to form a mixture, and processing the mixture by membrane extrusion followed by dialysis to obtain siCCR5-loaded engineered exosomes.

2. The method of claim 1, wherein the organic solvent comprises a chloroform:methanol mixture at a volume ratio of 9:1, and the molar ratio of DLin-MC3-DMA:cholesterol:DOPC is 25-34.5:35-65:1.5-40.

3. The method of claim 1, wherein hydrating the lipid film comprises mixing the lipid film with PBS at a mass ratio of 100:3, and wherein the mass ratio of siCCR5:hUCMSC-EVs:lipid film is 2:1:50.

4. The method of claim 1, wherein the mixture is incubated at a temperature of 40-50° C. during extrusion.

5. The method of claim 1, wherein the siCCR5 comprises a sense strand having the sequence of SEQ ID NO:1 and an antisense strand having the sequence of SEQ ID NO:2 and is configured to knock down CCR5 expression.

6. The method of claim 1, wherein said extruding comprises sequentially extruding the mixture through filters having pore sizes of 1.0 μm, 0.1 μm, and 0.05 μm.

7. The method of claim 6, wherein the filters are polycarbonate membrane filters and each extrusion step is repeated 2-5 times.

8. The method of claim 1, wherein dialyzing comprises dialyzing the preparation overnight at 4° C. using a membrane having a molecular-weight cutoff of 100 kDa and adjusting the pH to 7.2-7.4.

9. An engineered exosome prepared by the method of claim 1.

10. A method of treating Alzheimer's disease in a subject in need thereof, comprising administering to the subject a therapeutically effective amount of the engineered exosome of claim 9.
